(19) United States
(12) Patent Application Publication
(10) Pub. No.: US 2021/0145252 A1
(43) Pub. Date: May 20, 2021

(54) MEDICAL ROTATION MECHANISM, AND ENDOSCOPE DEVICE

(71) Applicant: OLYMPUS CORPORATION, Tokyo (JP)

(72) Inventor: Noriaki YAMANAKA, Tokyo (JP)

(73) Assignee: OLYMPUS CORPORATION, Tokyo (JP)

(21) Appl. No.: 17/142,344

(22) Filed: Jan. 6, 2021

Related U.S. Application Data
(63) Continuation of application No. PCT/JP2018/026062, filed on Jul. 10, 2018.

(51) Int. Cl. A61B 1/00; F16H 1/32

(52) U.S. Cl. CPC A61B 1/0016; F16H 1/32

(57) ABSTRACT

A medical rotation mechanism has a cylindrical force transmission member; a cylindrical driver disposed at an external side of the force transmission member and having a plurality of external teeth; and a cylindrical rotation member formed disposed at an external side of the driver and having a plurality of internal teeth, wherein the force transmission member has a cam portion configured to push the driver outwardly in a radial direction, a part of the cam portion in the circumferential direction having a larger length in the radial direction than that of the other part, the force transmission member is configured to transmit the force to the driver and move an inscribed engagement portion in the circumferential direction so as to rotate the rotation member, and the force transmission member has a roller configured to transmit a rotation force to the driver.

This application is a continuation application based on a PCT International Application No. PCT/JP2018/026062, filed on Jul. 10, 2018. The content of the PCT International Application is incorporated herein by reference.

### TECHNICAL FIELD

The present disclosure relates to a medical rotation mechanism and an endoscope device.

### BACKGROUND ART

A medical rotation mechanism for assisting operations of inserting a medical apparatus such as an endoscope device having an insertion portion into the luminous organ is known, wherein the insertion portion of the endoscope device has an imaging portion disposed at a distal end of the insertion portion configured to capture an image inside the luminous organ for observation.

In United States patent application, Publication No. 2012/0029281, an endoscope device having a medical rotation mechanism rotating around a longitudinal axis in an insertion portion is disclosed.

In Japanese Patent (Granted) Publication No. 5458224, a living body introduction apparatus having a rotation mechanism in an insertion portion is disclosed, wherein the rotation mechanism is configured to rotate a spiral-shaped fin. The living body introduction apparatus is configured to rotate the spiral-shaped fin disposed at an outside of the insertion portion by rotating the rotation mechanism connected with a shaft in the insertion portion. The living body introduction apparatus is configured to assist inserting the insertion portion into the luminous organ by the rotation of the spiral-shaped fin.

According to the living body introduction apparatus disclosed in Japanese Patent (Granted) Publication No. 5458224, it is necessary to secure a space for inserting treatment devices into the insertion portion of an endoscope apparatus while disposing a reduction drive having a high reduction ration in the rotation mechanism in the insertion portion.

### SUMMARY

According to an aspect of the present disclosure, a medical rotation mechanism has a force transmission member formed in a cylindrical shape and configured to transmit a force; a driver formed in a cylindrical shape and disposed at an external side of the force transmission member, the driver having a plurality of external teeth arrayed in a circumferential direction on an external circumferential surface of the driver; and a rotation member formed in a cylindrical shape and disposed at an external side of the driver, wherein the rotation member having a plurality of internal teeth arrayed in a circumferential direction on an internal circumferential surface of the rotation member. The force transmission member has a cam portion configured to push the driver outwardly in a radial direction, apart of the cam portion in the circumferential direction having a larger length in the radial direction than that of the other part in the circumferential direction of the cam portion. The force transmission member is configured to transmit the force to the driver and move an inscribed engagement portion in the circumferential direction so as to rotate the rotation member, wherein the inscribed engagement portion is a portion where the external teeth and the internal teeth inscribedly engage with each other in at least one location. The force transmission member has a roller configured to transmit a rotation force to the driver, wherein the roller is provided on the external circumferential surface of the force transmission member to be rotatably supported in the circumferential direction, and the roller is in contact with the driver.

According to another aspect of the present disclosure, a medical rotation mechanism has a force transmission member formed in a cylindrical shape and configured to transmit a force; a driver formed in a cylindrical shape and disposed at an external side of the force transmission member; a cover having elasticity and configured to cover the external surface of the driver; and a rotation member formed in a cylindrical shape and disposed at an external side of the driver. The force transmission member has a cam portion configured to push the driver outwardly in a radial direction, a part of the cam portion in the circumferential direction having a larger length in the radial direction than that of the other part in the circumferential direction of the cam portion. The driver has a plurality of external teeth which are arrayed in the circumferential direction and movable outwardly in a radial direction. The rotation member has a plurality of internal teeth arrayed in the circumferential direction on an internal circumferential surface of the rotation member. The cover is configured to press the plurality of external teeth inwardly in the radial direction. The cam portion of the force transmission member pushes part of the plurality of external teeth outwardly in the radial direction to make the plurality of external teeth and the plurality of internal teeth to inscribedly engage with each other. The force from the force transmission member is transmitted to the rotation member via the driver to rotate the rotation member.

According to a further aspect of the present disclosure, a medical rotation mechanism has a force transmission member formed in a cylindrical shape and configured to transmit a force; a driver formed in a cylindrical shape and having elasticity, the driver being disposed at an external side of the force transmission member; and a rotation member formed in a cylindrical shape and disposed at an external side of the driver, wherein the force transmission member has a cam portion configured to push the driver outwardly in a radial direction, a part of the cam portion in the circumferential direction having a larger length in the radial direction than that of an other part in the circumferential direction of the cam portion, the driver has a plurality of external teeth which are arrayed in the circumferential direction and movable outwardly in a radial direction, the rotation member has a plurality of internal teeth arrayed in the circumferential direction on an internal circumferential surface of the rotation member, the cam portion of the force transmission member pushes part of the plurality of external teeth outwardly in the radial direction to make the plurality of external teeth and the plurality of internal teeth to inscribedly engage with each other, and the force from the force transmission member is transmitted to the rotation member via the driver to rotate the rotation member.

### BRIEF DESCRIPTION OF DRAWINGS

FIG. 1 is a view showing a concept apparent configuration of an endoscope device having an insertion portion in which a medical rotation mechanism according to the first embodiment of the present disclosure is provided.

FIG. 2 is a perspective view showing the medical rotation mechanism with some parts detached.

FIG. 3 is a perspective view showing the medical rotation mechanism with some parts detached.

FIG. 4 is a perspective view showing the medical rotation mechanism at the time of attaching an external rotation cylinder of the medical rotation mechanism thereto.

FIG. 5 is a cross-sectional view showing a cross section A-A of the medical rotation mechanism.

FIG. 6 shows a cross-sectional view (a) of a driving member in the cross section A-A of the medical rotation mechanism and a side-view of the driving member.

FIG. 7 is a cross-sectional view showing a cross section B-B of the driving member.

FIG. 8 is a cross-sectional view showing a cross section B-B of the driving member.

FIG. 9 is a cross-sectional view of a medical rotation mechanism configured to transmit a rotation force to the external rotation cylinder.

FIG. 10 is a cross-sectional view of the medical rotation mechanism configured to transmit the rotation force to the external rotation cylinder.

FIG. 11 is a cross-sectional view of a medical rotation mechanism configured to transmit the rotation force to the external rotation cylinder.

FIG. 12 is a cross-sectional view of a medical rotation mechanism configured to transmit the rotation force to the external rotation cylinder.

FIG. 13 is a cross-sectional view of a modification example of the medical rotation mechanism.

FIG. 14 is a cross-sectional view of a medical rotation mechanism according to a second embodiment of the present disclosure.

FIG. 15 is a cross-sectional view of a driving member of the medical rotation mechanism.

FIG. 16 is a cross-sectional view of a modification example of the medical rotation mechanism.

FIG. 17 is a cross-sectional view of a modification example of the medical rotation mechanism.

FIG. 18 is a cross-sectional view of a medical rotation mechanism according to a third embodiment of the present disclosure.

FIG. 19 is a cross-sectional view of a modification example of the medical rotation mechanism.

FIG. 20 is a cross-sectional view of a modification example of the medical rotation mechanism.

FIG. 21 is a side-view showing a treatment device provided in a medical rotation mechanism according to a fourth embodiment of the present disclosure.

### DESCRIPTION OF EMBODIMENT

### First Embodiment

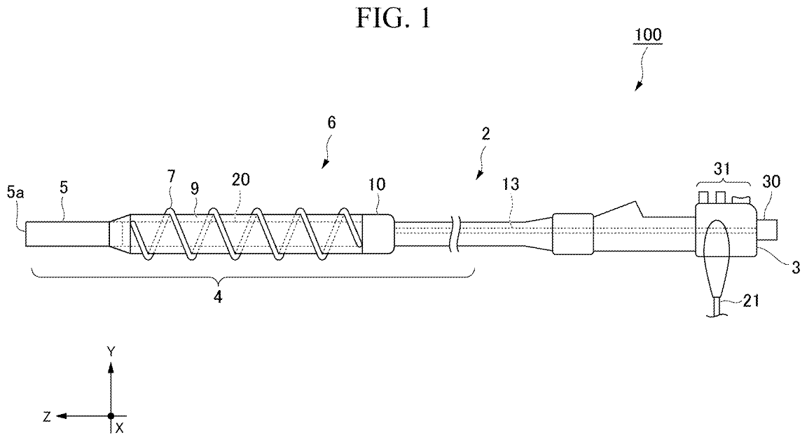

A first embodiment of the present disclosure will be described with reference to FIG. 1 to FIG. 12.

FIG. 1 is a view showing a concept apparent configuration of an endoscope device 100 having an insertion portion in which a medical rotation mechanism 10 according to the present embodiment is disposed. As shown in FIG. 1, the endoscope device 100 has an insertion portion 2 inserted into the luminous organ of a living body and an operation portion (operator) 3 provided at a proximal end side of the insertion portion 2.

As shown in FIG. 1, the insertion portion 2 has an elongated insertion portion main body 4 extending along a longitudinal axial direction of the insertion portion 2, a bending portion 5 disposed at a distal end side of the insertion portion main body 4, a living body insertion mechanism 6, and a medical rotation mechanism 10.

The bending portion 5 is an elongated member which bends following a curvature of the luminous organ. An imaging portion (imaging sensor) not shown in figures is disposed in a distal end portion 5a of the bending portion 5. A channel 20 is formed in the insertion portion 2 as a passage (internal cavity) from the distal end portion 5a over the whole length of the insertion portion 2. Treatment devices such as a high-frequency knife, a pair of holding forceps and the like are inserted into the channel 20.

The living body insertion mechanism 6 is a tubular member engaged to the insertion portion main body 4 or the bending portion 5 so as to form a gap between an outer circumference of the insertion portion main body 4 or the bending portion 5 and the living body insertion mechanism 6, and the living body insertion mechanism 6 is attachable to and detachable from the medical rotation mechanism 10. The living body insertion mechanism 6 has a fin 7 configured to function as an advancing portion and a retracting portion, a spiral tube (introduction advancing portion) 9 configured to rotate with a longitudinal axis as a rotation center so as to function as an introduction advancing member.

The fin 7 is spirally wound on an outer circumference of the spiral tube 9. The living body insertion mechanism 6 advances in the luminous organ by rotating the spiral tube 9 in a direction opposite to the spiral direction when winding the fin 7 on the outer circumference of the spiral tube 9. On the other hand, the living body insertion mechanism 6 retracts in the luminous organ by rotating the spiral tube 9 in the same direction with the spiral direction when winding the fin 7 on the outer circumference of the spiral tube 9.

The spiral tube 9 is formed from a material (for example, a rubber material or a resin material) having flexibility or formed in a structure so as to follow the bending of the bending portion 5. A distal end side of the spiral tube 9 is formed in a tapered shape so as to be easy to be inserted into the luminous organ.

The living body insertion mechanism 6 is a throwaway component (disposable component) being attachable to and detachable from the medical rotation mechanism 10, and the living body insertion mechanism is exchangeable per each treatment so as to prevent infection.

The medical rotation mechanism 10 is configured to rotate the spiral tube 9 around the longitudinal axis of the insertion portion 2 as the rotation center so as to assist inserting the insertion portion 2 into the luminous organ. The medical rotation mechanism 10 may rotate the spiral tube 9 in both directions i.e. the clock-wise direction and the counter-clock-wise direction (CW direction and CCW direction).

The medical rotation mechanism 10 is connected to a first end of a shaft 13 being inserted through the inside of the insertion portion 2, and a second end of the shaft 13 is connected to a motor disposed in the operation portion 3 which is not shown in figures. The motor is configured to rotate the shaft 13 around the longitudinal axis as the rotation center so as to rotate part of the medical rotation mechanism 10.

In the operation portion 30, a knob 30 and a switch 31 configured to perform various operations including the bending operation of the bending portion 5 and the rotation of the medical rotation mechanism 10 are disposed.

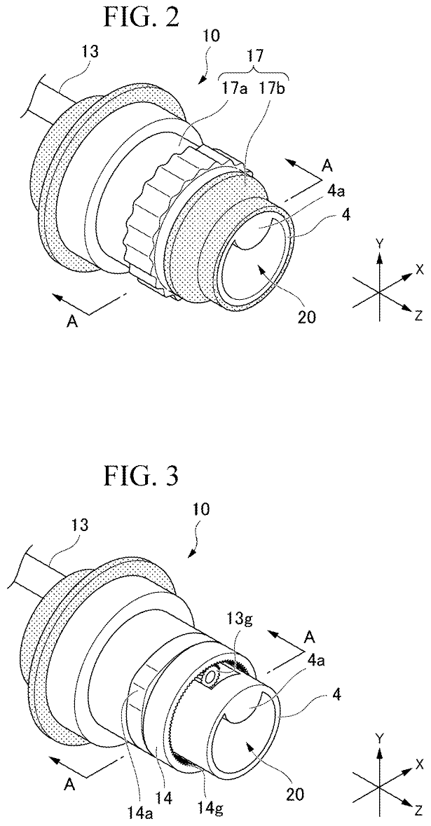

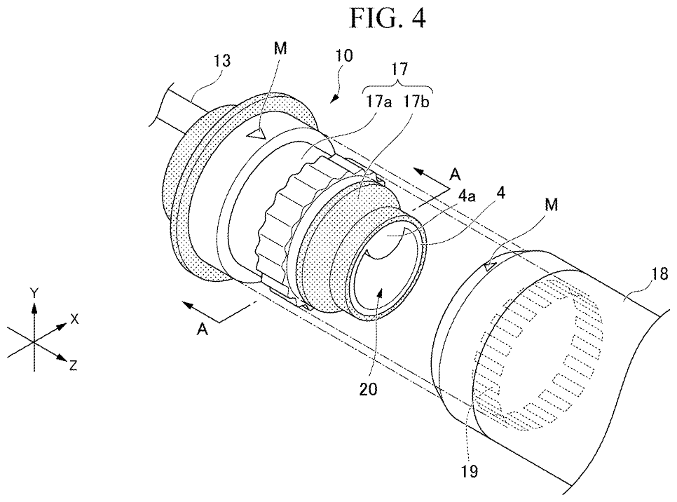

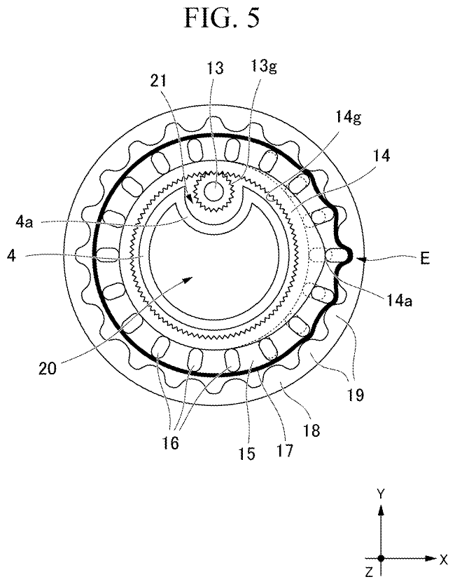

FIG. 2 is a perspective view showing the medical rotation mechanism 10 by detaching an external rotation cylinder 18 from the medical rotation mechanism 10. FIG. 3 is a perspective view showing the medical rotation mechanism 10 by detaching a driving member (driver) 15, a covering member 17, and the external rotation cylinder 18. FIG. 4 is a perspective view showing the medical rotation mechanism 10 at the time of attaching the external rotation cylinder 18 to the medical rotation mechanism 10. FIG. 5 is a cross-sectional view showing a cross section A-A of the medical rotation mechanism 10 shown in FIG. 2. In the description below, the cross section A-A is referred to an XY plane, and a longitudinal direction of the insertion portion 2 is referred to a Z-axis direction.

As shown in FIG. 5, the medical rotation mechanism 10 has a driving gear 13g connected to the shaft 13, an internal rotation cylinder (force transmission member) 14 which is inscribedly engaged with the driving gear 13g, a driving member (driver) 15, a covering member 17 configured to cover the driving member 15, and an external rotation cylinder (rotation member) 18.

The shaft 13 is disposed inside a gear cylinder 4a forming a cavity 21 which is separated from the channel 20 of the insertion portion 2. The cavity 21 forms a passage from the proximal end of the insertion portion 2 to the medical rotation mechanism 10. As shown in FIG. 3 and FIG. 5, the cavity 21 communicates with the internal space of the internal rotation cylinder 14 at least in the cross section A-A. An end portion of the shaft 13 is connected to the driving gear 13g.

As shown in FIG. 5, the internal rotation cylinder (force transmission member) 14 is a cylindrical member having a transmission gear 14g arrayed in a circumferential direction on an internal circumferential surface of the internal rotation cylinder 14. The transmission gear 14g is a gear inscribed engaged with the driving gear 13g. The internal rotation cylinder 14 is supported by the insertion portion main body 4 so as to be rotatable around the longitudinal axis as the rotation center. The internal rotation cylinder 14 rotates around the longitudinal axis as the rotation center in accordance with the rotation of the driving gear 13g with which the transmission gear 14g is inscribed engaged.

As shown in FIG. 3, the internal rotation cylinder 14 has a cam portion 14a, wherein a part of the cam portion 14a in a circumferential direction has a length in a radial direction larger than that of the other part of the cam portion 14a. When the internal rotation cylinder 14 rotates around the longitudinal axis as the rotation center, the cam portion 14a moves in the circumferential direction. The internal rotation cylinder 14 is configured to cause the cam portion 14a to come in contact with the driving member 15 which is disposed at an outside of the internal rotation cylinder 14 so as to transmit a rotation force around the longitudinal axis of the internal rotation cylinder 14 as the rotation center to the driving member 15.

In the description below, the part having the larger length in the radial direction than other part of the cam portion 14a in the circumferential direction is assigned as “a distal end of the cam portion 14a”.

As shown in FIG. 5, the driving member 15 is disposed at the outside in the radial direction of the internal rotation cylinder 14, and the driving member 15 is a cylindrical member having a plurality of external teeth 16 arrayed in the circumferential direction. The driving member 15 is supported so as to be unable to rotate relative to the insertion portion main body 4.

As shown in FIG. 5, the covering member 17 is an elastic member disposed between the driving member 15 and the external rotation cylinder 18, and the covering member 17 is configured to cover the driving member 15 to make the inside of the covering member 17 to be watertight.

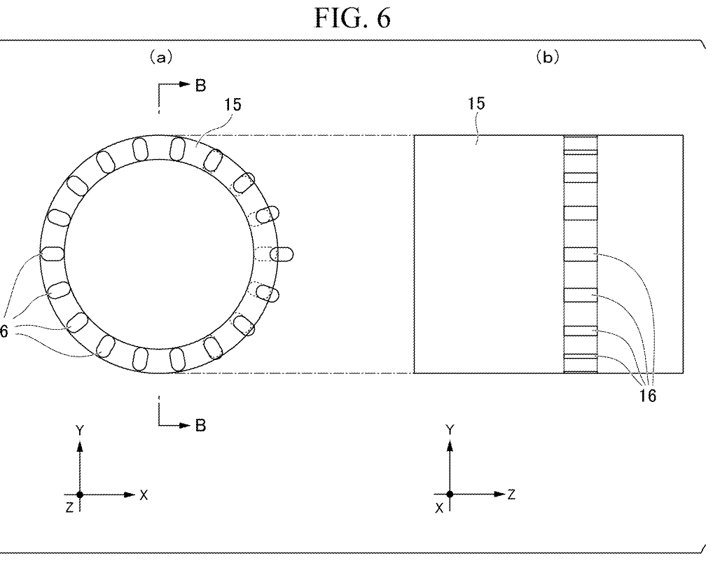

In FIG. 6, a part (a) of FIG. 6 shows a cross-sectional view of the driving member 15 in the cross section A-A of the medical rotation mechanism 10 shown in FIG. 2. A part (b) of FIG. 6 is a side-view of the driving member 15.

As shown in the part (a) of FIG. 6, the plurality of external teeth 16 are configured to support the driving member 15 such that the driving member 15 is movable in the radial direction. The plurality of external teeth 16 are arranged at uniform distances in the circumferential direction. As shown in the part (b) of FIG. 6, a length of the plurality of external teeth 16 in the Z-axis is shorter than the length of the driving member 15. In the Z-axis, the external teeth 16 is disposed at an overlapping position of the cam portion 14a of the internal rotation cylinder 14.

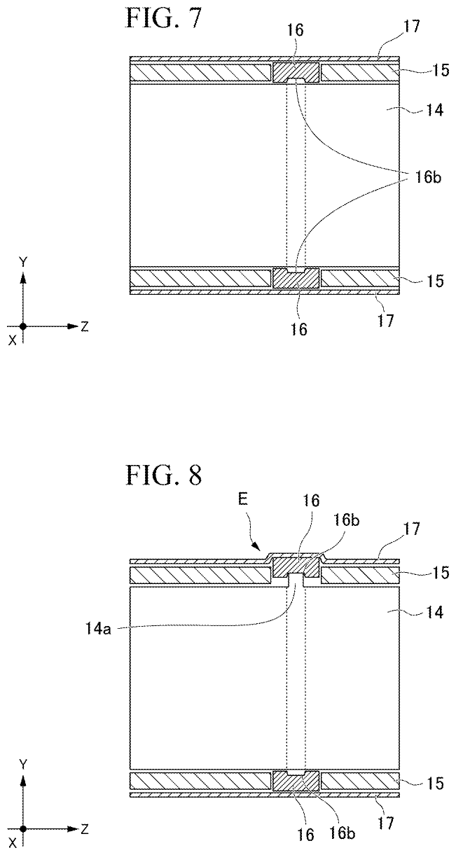

FIG. 7 and FIG. 8 are cross-sectional views of the cross section B-B of the driving member 15 shown in the part (a) of FIG. 6.

As shown in FIG. 7, in a case in which the force is not transmitted by the cam portion 14a from the internal side of the radial direction, the external teeth 16 are pressed by the covering member 17 so as to not to protrude toward the external side of the driving member 15. On the other hand, as shown in FIG. 8, the external teeth 16 are pushed out toward the external side of the driving member 15 by the cam portion 14a which comes in contact with the external tenth 16 from the internal side in the radial direction. As shown in FIG. 8, the external teeth 16 which are pushed out by the cam portion 14a may press the covering member 17 such that the external teeth 16 protrude toward the external side in the radial direction of the driving member 15.

As shown in FIG. 7 and FIG. 8, the external teeth 16 have fitting concave portions 16b formed in the internal side of the radial direction and configured for the cam portion 14a to fit in. When the cam portion 14a comes in contact with the external teeth 16, the distal portion of the cam portion 14a comes in contact with a bottom surface of the fitting concave portion 16b such that the cam portion 14a may straightly push out the external teeth 16 in the radial direction.

As shown in FIG. 2, the covering member 17 is configured to have a first covering member 17a formed at a proximal end side and formed from the resin material and a second covering member 17b formed at a distal end side and formed from the rubber material.

The first covering member 17a is a portion which comes in contact with the external teeth 16 of the driving member 15 and pushed out by the external teeth 16, and it is desirable to form the first covering member 17a from a softer material such as the resin than the material of the driving member 15. The first covering member 17a, particularly the part pushed out by the external teeth 16 may be formed from the elastic member such as the rubber.

The second covering member 17b is a member configured at the distal end of the first covering member 17a so as to make the inside of the covering member 17 to be watertight. The second covering member 17b has a tapered shape whose outer diameter becomes smaller toward the distal end, and the distal end of the second covering member 17b is configured to fit closely with the insertion portion main body 4. It is desirable to form the second covering member 17b from the elastic member such as the rubber or the like to make the second covering member 17b easy to fit closely with the insertion portion main body 4.

As shown in FIG. 5, the external rotation cylinder (rotation member) 18 is disposed at the external side in the radial direction of the driving member 15, and the external rotation cylinder 18 is a cylindrical member having a plurality of internal teeth 19 arrayed in the circumferential direction on an internal surface of the external rotation cylinder 18. The external rotation cylinder 18 is supported so as to be rotatable around the longitudinal axis as the rotation center relative to the insertion portion main body 4. The external rotation cylinder 18 is connected with the spiral tube 9 such that when the external rotation cylinder 18 rotates around the longitudinal axis as the rotation center, the spiral tube 9 also rotates around the longitudinal axis as the rotation center. The external rotation cylinder 18 and the spiral tube 9 may be integrally formed.

As shown in FIG. 4, the external rotation cylinder 18 is attached to the endoscope device 100 at the external side of the covering member 17 to be attachable to and detachable from the endoscope device 100. The external rotation cylinder 18 is attached to the endoscope device 100 in a rotatable state around the longitudinal axis as the rotation center by fitting the external rotation cylinder 18 to the external circumference of the covering member 17 to forma gap between the external rotation cylinder 18 and the covering member 17. The external rotation cylinder 18 is a disposable member that is attachable to and detachable from the endoscope device 100, and the external rotation cylinder 18 is exchangeable by each treatment so as to prevent infection.

As shown in FIG. 4, the medical rotation mechanism 10 has a marker M for indicating an attachment position for the external rotation cylinder 18. A surgeon can rapidly attach the external rotation cylinder 18 to the correct position by viewing the marker M.

As shown in FIG. 5, the internal teeth 19 are arranged equally in the circumferential direction on the internal circumferential surface of the external rotation cylinder 18, and a cycloid curve or a cycloid parallel curve is formed in the circumferential direction on the internal circumferential surface including the internal teeth 19. As shown in FIG. 5, the number of internal teeth 19 included in the external rotation cylinder 18 is 20. On the other hand, the number of external teeth 16 included in the driving member 15 is 18. In other words, the number of internal teeth 19 is more than the number of external teeth 16.

The number of external teeth 16 included in the driving member 15 and the number of internal teeth 19 included in the external rotation cylinder 18 are not limited thereto. The number of internal teeth 19 only has to be more than the number of external teeth 16. For example, the number of external teeth 16 may be a value calculated by subtracting one from the number of internal teeth 19. Specifically, the number of external teeth 16 may be the value calculated by subtracting 1 from 20, that is, the number of external teeth 16 may be 19.

As shown in FIG. 5, due to the contact by the cam portion 14a of the internal rotation cylinder 14, the external teeth 16 which are pushed out toward the external side in the radial direction of the driving member 15 engage with the internal teeth 19 in an inscribed form. The internal rotation cylinder 14 is configured to form the inscribed engagement between the external teeth 16 and the internal teeth 19 and move a portion where the inscribed engagement between the external teeth 16 and the internal teeth 19 is formed (hereinafter, refers to “inscribed engagement portion E”) in the circumferential direction such that the rotation force of the internal rotation cylinder 14 around the longitudinal axis as the rotation center is transmitted to the external rotation cylinder 18 via the driving member 15. As a result, the external rotation cylinder 18 rotates around the longitudinal axis as the rotation center.

The length of the internal teeth 19 in the Z-axis direction is shorter than the length of the external teeth 16. At the time of attaching the external rotation cylinder 18 to the covering member 17, the external teeth 16 are accommodated in valley portions of the internal teeth 19 in the Z-axis direction. At the time when the external teeth 16 and the internal teeth 19 inscribedly engage with each other, the force is uniformly transmitted from the external teeth 16 to the internal teeth 19. As a result, a product life cycle of the external teeth 16 which is not a disposable member may be extended.

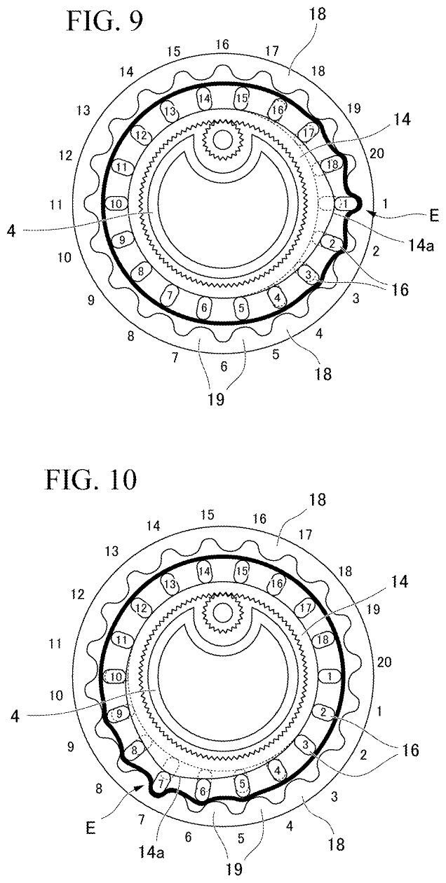

Next, effects of the medical rotation mechanism 10 will be descried referring to FIGS. 9-12. FIGS. 9-12 are cross-sectional views of the medical rotation mechanism 10 showing aspects of transmitting the rotation force to the external rotation cylinder 18 by the internal rotation cylinder 14 via the driving member 15.

In the medical rotation mechanism 10, as shown in FIGS. 9-12, the driving member 15 having the external teeth 16 and the external rotation cylinder 18 function as an external gear and an internal gear engaging with each other in the inscribed form at one location. The number of internal teeth 19 is more than the number of external teeth 16 such that the medical rotation mechanism functions as a deceleration mechanism. As shown in FIG. 9, a reduction ratio of the medical rotation mechanism 10 according to the present embodiment may be calculated by a numerical formula (the number of internal teeth 19−the number of external teeth 16)/the number of internal teeth 19, specifically, the reduction ratio equals to (20−18)/20= 1/10.

In FIG. 9, the external teeth 16 and the internal teeth 19 engaging with each other in the inscribed form are designated as the external teeth 16 having a serial number “1” and the internal teeth 19 having a serial number “1”. As shown in FIG. 9, each of the external teeth 16 and the internal teeth 19 are assigned to serial numbers continuing from “1” along a clock-wise sequence in the circumferential direction. With respect to the internal teeth 19, the valley portion with which the external teeth 16 engage is assigned to a serial number.

In FIG. 9, the external teeth 16 with the serial number “1” and the internal teeth 19 with the serial number “1” inscribedly engage with each other. In this state, the internal rotation cylinder 14 is rotated in the clock-wise direction around the longitudinal axis as the rotation center so as to move the cam portion 14a in the clock-wise direction. As a result, the cam portion 14a moves in the circumferential direction such that the distal end of the cam portion 14a comes in contact with the external teeth 16 having the serial number “2”. The external teeth 16 having the serial number “2” that comes in contact with the distal end of the cam portion 14a is pushed out by the cam portion 14a so as to protrude outwardly in the radial direction of the driving member 15 compared with other external teeth 16.

On the other hand, the distal end of the cam portion 14a gradually loses contact with the external teeth 16 having the serial number “1”, and the external teeth 16 having the serial number “1” is pressed by the covering member 17 so as not to protrude toward the external side in the radial direction of the driving member 15. As a result, the external teeth 16 having the serial number “1” and the internal teeth 19 having the serial number “1” do not inscribedly engage with each other.

Next, the external teeth 16 having the serial number “2” approaches the internal teeth 19 having the serial number “2” in the protrusion procedure. Since the external teeth 16 having the serial number “2” approaches the internal teeth 19 having the serial number “2”, the external rotation cylinder 18 having the internal surface on which the cycloid curve or the cycloid parallel curve is formed in the circumferential direction rotates in the clock-wise direction around the longitudinal axis as the rotation center. As a result, the external teeth 16 having the serial number “2” and the internal teeth 19 having the serial number “2” further approach each other and inscribedly engage with each other.

By rotating the internal rotation cylinder 14 around the longitudinal axis as the rotation center, the cam portion 14a moves in the circumferential direction such that the adjacent external teeth 16 is sequentially pushed out toward the external side in the radial direction of the driving member 15. As a result, the inscribed engagement portion E where the external teeth 16 and the internal teeth 19 inscribedly engage with each other moves in the circumferential direction.

FIG. 10 is a cross-sectional view of the medical rotation mechanism 10 showing that the internal rotation cylinder 14 further rotates and the inscribed engagement portion E is the portion where the external teeth 16 having the serial number “7” and the internal teeth 19 having the serial number “7” engage with each other. Compared with the external rotation cylinder 18 shown in FIG. 9, the external rotation cylinder 18 shown in FIG. 10 rotates in the clock-wise direction around the longitudinal axis as the rotation center.

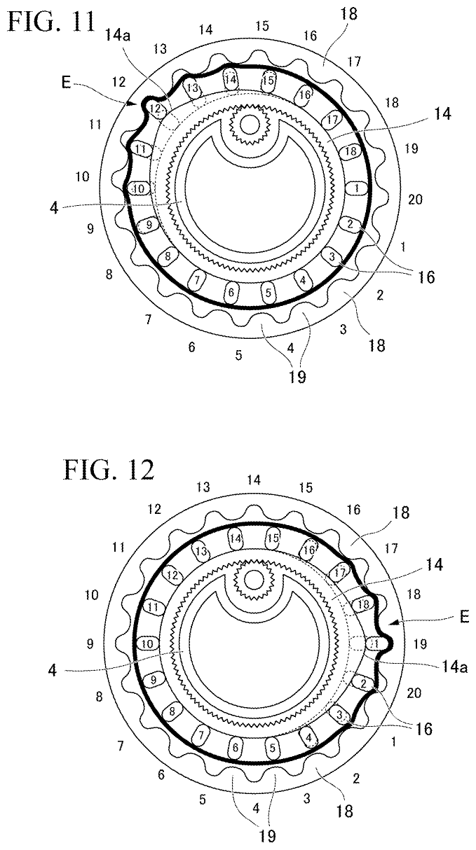

FIG. 11 is a cross-sectional view of the medical rotation mechanism 10 showing that the internal rotation cylinder 14 further rotates and the inscribed engagement portion E is the portion where the external teeth 16 having the serial number “12” and the internal teeth 19 having the serial number “12” engage with each other. Compared with the external rotation cylinder 18 shown in FIG. 10, the external rotation cylinder 18 shown in FIG. 11 rotates in the clock-wise direction around the longitudinal axis as the rotation center.

FIG. 12 is a cross-sectional view of the medical rotation mechanism 10 showing that the internal rotation cylinder 14 further rotates at 360 degrees. In the medical rotation mechanism 10 rotating at 360 degrees, the inscribed engagement portion E becomes the portion where the external teeth 16 having the serial number “1” and the internal teeth 19 having the serial number “19” engage with each other. Even if the internal rotation cylinder 14 has rotated by 360 degrees around the longitudinal axis as the rotation center, the external rotation cylinder 18 does not rotate by 360 degrees. In other words, the medical rotation mechanism 10 functions as the deceleration mechanism.

As shown in FIGS. 9-12, the rotation center of the internal rotation cylinder 14 and the rotation center of the external rotation cylinder 18 coincide with each other.

According to the medical rotation mechanism 10 according to the present embodiment, it is possible to secure enough space in the channel 20 for inserting the treatment devices or the like inside the insertion portion 2 and configure the deceleration mechanism in the insertion portion 2.

According to the medical rotation mechanism 10 according to the present embodiment, since a large reduction ratio is achieved, it is possible to maintain the driving force of the external rotation cylinder 18 and reduce the load applied to the internal rotation cylinder 14. As a result, it is possible to reduce the size and the diameter of the member in the medical rotation mechanism 10.

According to the medical rotation mechanism 10 according to the present embodiment, the rotation center of the internal rotation cylinder 14 and the rotation center of the external rotation cylinder 18 coincide with each other. Accordingly, when the surgeon inserts the insertion portion 2 into the luminous organ, it is possible to rotate the spiral tube 9 around the longitudinal axis as the rotation center such that it is easy for the surgeon to handle.

According to the medical rotation mechanism 10 according to the present embodiment, the external teeth 16 of the driving member 15 engages with the internal teeth 19 of the external rotation cylinder 18 via the elastic member of the covering member 17. The covering member 17 has flexibility except for the part being pushed out by the external teeth 16. The covering member 17 having the flexibility is configured to improve the stability of the engagement between the external teeth 16 and the internal teeth 19 and improve the transmission efficiency of the rotation force.

The first embodiment of the present disclosure has been described above, however, technical scope of the present disclosure is not limited to the present embodiment and the application examples. The configuration elements disclosed in the above-described first embodiment and the following modification examples may be suitably combined to realize the configuration.

### First Modification Example

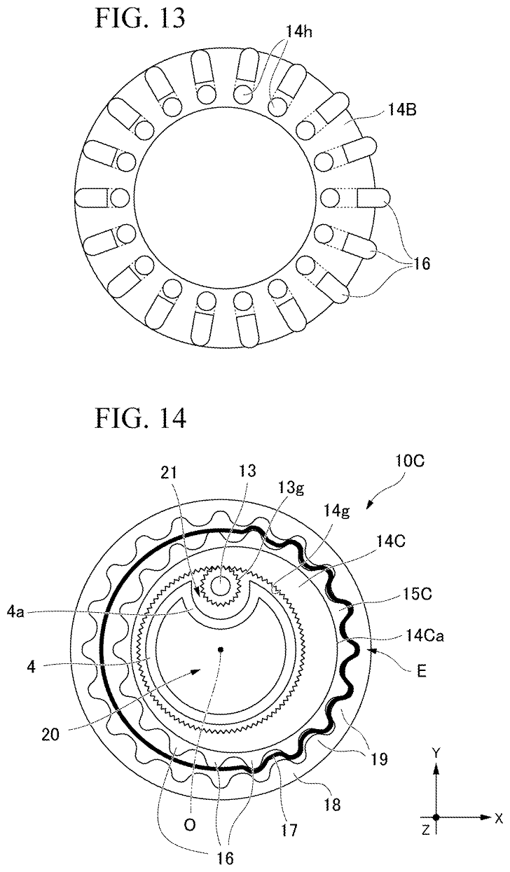

According to the above-described embodiment, the internal rotation cylinder (force transmission member) 14 rotates around the longitudinal axis as the rotation center to transmit the force to the driving member 15; however, the aspect of the force transmission of the internal rotation cylinder (force transmission member) 14 is not limited thereto. FIG. 13 is a cross-sectional view of part of a medical rotation mechanism 10B presented as a modification example of the medical rotation mechanism 10. The medical rotation mechanism 10B has a force transmission member 14B functioning as both of an internal rotation cylinder and a driving member. As shown in FIG. 13, the force transmission member 14B has a plurality of force transmission holes 14h. The force is transmitted from the operation portion 3 by a fluid (liquid or gas), a wire or the like through the plurality of force transmission holes 14h so as to move the external teeth 16 formed in the force transmission member 14B outwardly in the radial direction. By suitably controlling the external teeth 16 moved outwardly in the radial direction, it is possible to move the inscribed engagement portion E where the external teeth 16 and the internal teeth 19 inscribedly engage with each other in the circumferential direction so as to transmit the rotation force to the external rotation cylinder 18 and rotate the external rotation cylinder 18.

### Second Modification Example

According to the above-described embodiment, it is described that the cycloid curve or the cycloid parallel curve is formed in the circumferential direction on the internal circumferential surface of the external rotation cylinder 18 including the internal teeth 19; however, the embodiment of the internal surface of the external rotation cylinder (rotation member) is not limited thereto. A trochoid curve or a trochoid parallel curve may be formed in the circumferential direction on the internal circumferential surface of the external rotation cylinder (rotation member).

### Second Embodiment

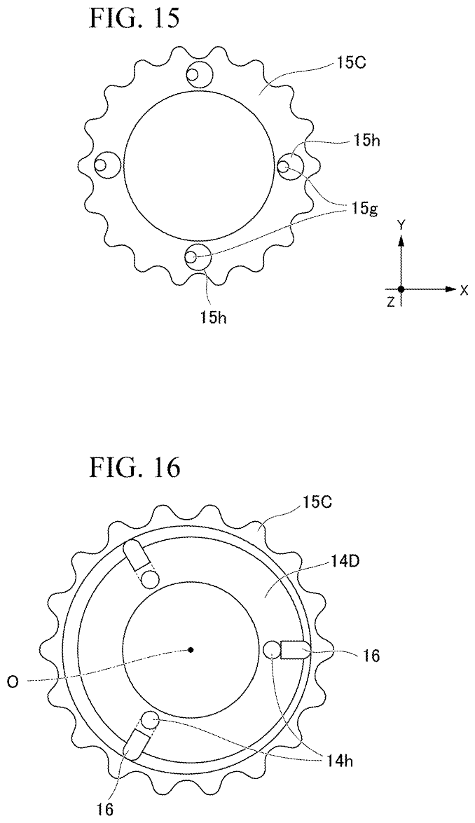

A second embodiment of the present disclosure will be described with reference to FIGS. 14-15. In the following description, the configurations having the same or similar functions with respect to the embodiment described above will be designated with the same reference sign and a redundant description will be omitted. The present embodiment is different from the first embodiment in that configurations of the internal rotation cylinder (force transmission member) and the driving member of the medical rotation mechanism are different.

FIG. 14 is a cross-sectional view of a medical rotation mechanism 100 according to the present embodiment.

As shown in FIG. 4, the medical rotation mechanism 100 has a driving gear 13g connected with the shaft 13, an internal rotation cylinder (force transmission member) 14C engaged with the driving gear 13g in the inscribed form, a driving member 15C, the covering member 17 covering the driving member 15C, and the external rotation cylinder (rotation member) 18.

As shown in FIG. 14, similar to the internal rotation cylinder 14, the internal rotation cylinder (force transmission member) 14C has a cam portion 14Ca, wherein a part of the cam portion 14Ca in the circumferential direction has a length in a radial direction larger than that of the other part of the cam portion 14Ca. The cam portion 14Ca moves in the circumferential direction by when the internal rotation cylinder 14C rotates around the longitudinal axis as the rotation center. The internal rotation cylinder 14C is configured to cause the cam portion 14Ca to come in contact with the driving member 15C which is disposed at an outside of the internal rotation cylinder 14C so as to transmit a rotation force around the longitudinal axis of the internal rotation cylinder 14C as the rotation center to the driving member 15.

The driving member 15C is a cylindrical member having external teeth 16C on the external circumferential surface of the driving member 15C. As shown in FIG. 14, the external teeth 16C are arranged equally in the circumferential direction on the external circumferential surface of the driving member 15C, and a cycloid curve or a cycloid parallel curve is formed in the circumferential direction on the external circumferential surface including the external teeth 16. The number of external teeth 16C is 18 that is equal to the number of external teeth 16.

FIG. 15 is a cross-sectional view of the driving member 15C.

Four penetration holes 15h are equally formed in the circumferential direction in the driving member 15C, and the penetration holes 15h are formed in the longitudinal direction of the driving member 15C. Restricting columnar members 15g are inserted into the penetration holes 15h respectively, and the restricting columnar members 15g are fixed to the insertion portion main body 4 such that a relative distance between each of the restricting columnar members 15g and the insertion portion main body 4 is the same. A movement amount of the driving member 15C in the X-Y direction is restricted by the restricting columnar members 15g. As a result, the rotation of the driving member 15C is restricted by the restricting columnar members 15g.

Due to a contact of the cam portion 14Ca of the internal rotation cylinder 14, the external teeth 16 of the driving member 15C which has the farthest distance from the rotation center axis O of the internal rotation cylinder 14C engages with the internal teeth in the inscribed form. The internal rotation cylinder 14C is configured to make the external teeth 16C and the internal teeth 19 to inscribedly engage with each other and move the inscribed engagement portion E where the external teeth 16 and the internal teeth 19 inscribedly engage with each other in the circumferential direction so as to transmit the rotation force around the longitudinal axis of the internal rotation cylinder 14C as the rotation center to the external rotation cylinder 18 via the driving member 15C. As a result, the external rotation cylinder 18 rotates around the longitudinal axis as the rotation center.

As shown in FIG. 14, the rotation center of the internal rotation cylinder 14C and the rotation center of the external rotation cylinder 18 coincide with each other.

According to the medical rotation mechanism 100 according to the present embodiment, similar to the first embodiment, it is possible to secure enough space in the channel 20 for inserting the treatment devices or the like inside the insertion portion 2 and configure the deceleration mechanism in the insertion portion 2. The driving member 15C has a relative simple configuration compared with the driving member 15 such that it is possible to reduce the number of necessary members.

The second embodiment of the present disclosure has been described above referring to figures, however, technical scope of the present disclosure is not limited to the present embodiment and the application examples. The configuration elements disclosed in the above-described second embodiment and the following modification examples may be suitably combined to realize the configuration.

### Third Modification Example

According to the above-described embodiment, the internal rotation cylinder (force transmission member) 14C rotates around the longitudinal axis as the rotation center to transmit the force to the driving member 15C; however, the aspect of the internal rotation cylinder (force transmission member) 14C is not limited thereto. FIG. 16 is a cross-sectional view of part of a medical rotation mechanism 10D presented as a modification example of the medical rotation mechanism 100. The medical rotation mechanism 10D has a force transmission member 14D. As shown in FIG. 16, the force transmission member 14D has three external teeth 16 and three force transmission holes 14h. The force is transmitted from the operation portion 3 by a fluid (liquid or gas), a wire or the like through the force transmission holes 14h so as to move the external teeth 16 formed in the force transmission member 14D outwardly in the radial direction. The external teeth 16C of the driving member 15C whose distance from the rotation center axis O of the internal rotation cylinder 14D becomes the farthest engages with the internal teeth 19 in the inscribed form. By suitably controlling the external teeth 16 moved outwardly in the radial direction, it is possible to move the inscribed engagement portion E where the external teeth 16 and the internal teeth 19 inscribedly engage with each other in the circumferential direction so as to transmit the rotation force to the external rotation cylinder 18 and rotate the external rotation cylinder 18.

### Modification Example 4

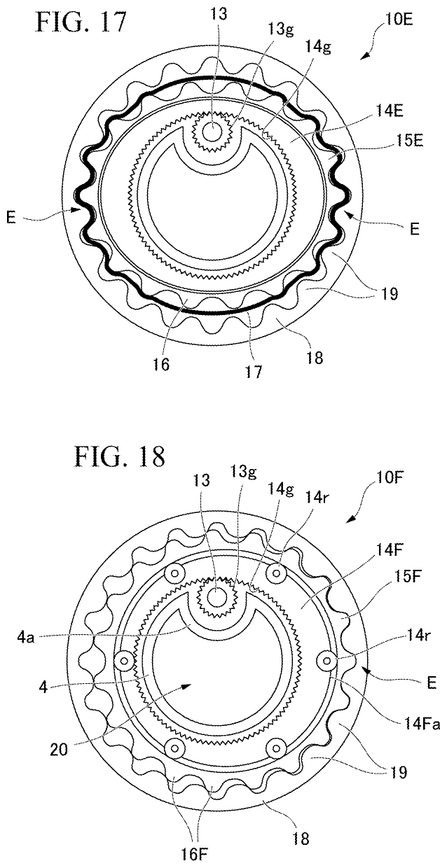

According to the above-described embodiment, the driving member 15 having the external teeth 16 and the external rotation cylinder 18 having the internal teeth 19 engage with each other in one location; however, aspects of the engagement are not limited thereto. FIG. 17 is a cross-sectional view of a medical rotation mechanism 10E as a modification example of the medical rotation mechanism 100. The medical rotation mechanism 10E has an elliptical force transmission member 14E and a driving member 15E in the X-Y plane. The driving member 15E engages with the external rotation cylinder 18 in the inscribed form at two locations via the covering member 17. Once the external teeth and the internal teeth engage with each other in at least one location, it is possible to transmit the rotation force to the external rotation cylinder 18 to rotate the external rotation cylinder 18.

### Modification Example 5

According to the above-described embodiment, it is described that the cycloid curve or the cycloid parallel curve is formed in the circumferential direction on the external circumferential surface including the external teeth 16C of the driving member 15C; however, the aspect of the external surface of the driving member is not limited thereto. A trochoid curve or a trochoid parallel curve may be formed in the circumferential direction on the external surface of the driving member.

### Third Embodiment

A third embodiment of the present disclosure will be described with reference to FIG. 18. In the following description, the configurations having the same or similar functions with respect to the embodiment described above will be designated with the same reference sign and a redundant description will be omitted. The present embodiment is different from the above-described embodiments in that the driving member and the covering member of the medical rotation mechanism are integrally configured.

FIG. 18 is a cross-sectional view of a medical rotation mechanism 10F according to the present embodiment.

As shown in FIG. 18, the medical rotation mechanism 10F has a driving gear 13g connected to the shaft 13, an internal rotation cylinder (force transmission member) 14F engaged with the driving gear 13g in the inscribed form, a driving member 15F, and an external rotation cylinder (rotation member) 18.

As shown in FIG. 18, the internal rotation cylinder (force transmission member) 14F has a cam portion 14Fa, wherein a part of the cam portion 14Fa in a circumferential direction has a length in a radial direction larger than that of the other part of the cam portion 14Fa. When the internal rotation cylinder 14F rotates around the longitudinal axis as the rotation center, the cam portion 14Fa moves in the circumferential direction.

The internal rotation cylinder 14F has a plurality of rollers 14r. The plurality of rollers 14r are equally arranged in the circumferential direction. The internal rotation cylinder 14F is configured to make the cam portion 14Fa to come in contact with the driving member 15F disposed at the external side of the internal rotation cylinder 14F via the plurality of rollers 14r so as to transmit the rotation force around the longitudinal axis of the internal rotation cylinder 14F to the driving member 15F.

Similar to the covering member 17 according to the above-described embodiment, the driving member 15F is an elastic member, and the driving member 15F is configured to cover the internal rotation cylinder 14 so as to make the inside of the driving member 15F to be watertight. External teeth 16F are formed on the external circumferential surface of the driving member 15F. As shown in FIG. 18, the external teeth 16F are equally arranged on the external circumferential surface of the driving member 15F, and a cycloid curve or a cycloid parallel curve is formed in the circumferential direction on the internal circumferential surface including the external teeth 16F. The number of external teeth 16F is calculated by a numerical formula “the number of internal teeth 19-1” to be 19.

Due to the contact of the roller near the cam portion 14Fa of the internal rotation cylinder 14F, the external teeth 16 whose distance from the rotation center axis O of the internal rotation cylinder 14C becomes the farthest engage with the internal teeth 19 in the inscribed form. The internal rotation cylinder 14F is configured to make the external teeth 16F and the internal teeth 19 to inscribedly engage with each other to move the inscribed engagement portion E where the external teeth 16F and the internal teeth 19 inscribedly engage with each other in the circumferential direction so as to transmit the rotation force around the longitudinal axis of the internal rotation cylinder 14F to the external rotation cylinder 18 via the driving member 15F. As a result, the external rotation cylinder 18 rotates around the longitudinal axis as the rotation center.

The driving member 15F is formed from the elastic member such that a friction force between the internal rotation cylinder 14F and the driving member 15F becomes larger. Thus, it is possible to obstruct a smooth rotation operation of the driving member 15F; however, since the internal rotation cylinder 14F has the plurality of rollers 14r so as to maintain the smooth rotation operation of the driving member 15F.

According to the medical rotation mechanism 10F according to the present embodiment, similar to the first embodiment, it is possible to secure enough space in the channel 20 for inserting the treatment devices or the like inside the insertion portion 2 and configure the deceleration mechanism in the insertion portion 2.

According to the medical rotation mechanism 10F according to the present embodiment, even if the external rotation cylinder 18 is out of place during the treatment, the external teeth 16F formed from the elastic member and having a rounded outer peripheral surface comes in contact with the living tissues so as to reduce the risk of damaging the living tissues.

The third embodiment of the present disclosure has been described above, however, technical scope of the present disclosure is not limited to the present embodiment and the application examples. The configuration elements disclosed in the above-described third embodiment and the following modification examples may be suitably combined to realize the configuration.

### Modification Example 6

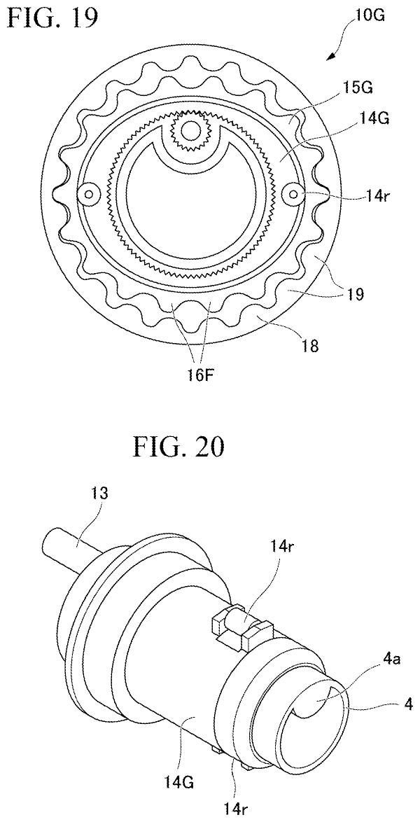

According to the above-described embodiment, the driving member 15F having the external teeth 16F and the external rotation cylinder 18 having the internal teeth 19 inscribedly engage with each other in one location; however, the aspect of the engagement is not limited thereto. FIG. 19 is a cross-sectional view of a medical rotation mechanism 10G as a modification example of the medical rotation mechanism 10F. FIG. 20 is a perspective view of the medical rotation mechanism 10G. The medical rotation mechanism 10G has an elliptical force transmission member 14G and a driving member 15G in the X-Y plane, and the forcer transmission member 14G and the driving member 15G are connected via the rollers 14r. The driving member 15G engages with the external rotation cylinder 18 in the inscribed form at two locations via a covering member. Once the external teeth 16F and the internal teeth 19 engage with each other in at least one location, it is possible to transmit the rotation force to the external rotation cylinder 18 to rotate the external rotation cylinder 18.

### Fourth Embodiment

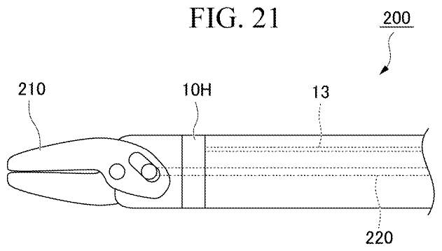

A fourth embodiment of the present disclosure will be described with reference to FIG. 21. In the following description, the configurations having the same or similar functions with respect to the embodiment described above will be designated with the same reference sign and a redundant description will be omitted. The present embodiment is different from the above-described embodiments in that the medical rotation mechanism is provided in a treatment device rather than the endoscope device.

FIG. 21 is a side-view of a treatment device 200 in which a medical rotation mechanism 10H according to the present embodiment is provided.

The treatment device 200 has a pair of forceps 210, an operation wire 220 for open-close operation, and the medical rotation mechanism 10H.

Similar to the medical rotation mechanism 10, the medical rotation mechanism 10H has the driving gear 13g connected to the shaft 13, the internal rotation cylinder (force transmission member) 14 engaging with the driving gear 13g in the inscribed form, the driving member 15, and the external rotation cylinder (rotation member) 18.

Similar to the endoscope 100 according to the first embodiment, according to the treatment device 200, the shaft 13 rotates such that the internal rotation cylinder 14 rotates around the longitudinal axis as the rotation center. The internal rotation cylinder 14 is configured to make the cam portion 14a to come in contact with the driving member 15 disposed at the external side of the internal rotation cylinder 14 so as to transmit the rotation force around the longitudinal axis of the internal rotation cylinder 14 as the rotation center to the driving member 15. The internal rotation cylinder 14 is configured to make the external teeth 16 and the internal teeth 19 to inscribedly engage with each other and move the inscribed engagement portion E where the external teeth 16 and the internal teeth 19 inscribedly engage with each other in the circumferential direction so as to transmit the rotation force around the longitudinal axis of the internal rotation cylinder 14 as the rotation center to the external rotation cylinder 18 via the driving member 15. As a result, the external rotation cylinder 18 rotates around the longitudinal axis as the rotation center.

The external rotation cylinder 18 is connected to the pair of forceps 210, the pair of forceps 210 rotate around the longitudinal axis as the rotation center by rotating the external rotation cylinder 18 around the longitudinal axis as the rotation center.

According to the medical rotation mechanism 10H according to the present embodiment, it is possible to provide a rotation mechanism having a deceleration mechanism in the treatment device 200 having a small diameter.

Several embodiments and modification examples of the present disclosure have been described above, however, technical scope of the present disclosure is not limited to the embodiment and the application examples. Additions, omissions, substitutions and other changes in the structure are possible without departing from the spirit of the present disclosure. The present disclosure is not limited to the above-described embodiments and is limited only by the accompanying claims.

What is claimed is:

1. A medical rotation mechanism, comprising:
a force transmission member formed in a cylindrical shape and configured to transmit a force;
a driver formed in a cylindrical shape and disposed at an external side of the force transmission member, the driver having a plurality of external teeth arrayed in a circumferential direction on an external circumferential surface of the driver; and
a rotation member formed in a cylindrical shape and disposed at an external side of the driver, wherein the rotation member having a plurality of internal teeth arrayed in a circumferential direction on an internal circumferential surface of the rotation member,
wherein the force transmission member has a cam portion configured to push the driver outwardly in a radial direction, a part of the cam portion in the circumferential direction having a larger length in the radial direction than that of an other part in the circumferential direction of the cam portion,
the force transmission member is configured to transmit the force to the driver and move an inscribed engagement portion in the circumferential direction so as to rotate the rotation member, wherein the inscribed engagement portion is a portion where the external teeth and the internal teeth engage with each other in at least one location, and
the force transmission member has a roller configured to transmit a rotation force to the driver, wherein the roller is provided on the external circumferential surface of the force transmission member to be rotatably supported in the circumferential direction, and the roller is in contact with the driver.

2. The medical rotation mechanism according to claim 1, wherein a number of plurality of internal teeth is more than a number of plurality of external teeth.

3. The medical rotation mechanism according to claim 1, wherein the force transmission member is configured to transmit the force to the driver by rotating around a longitudinal axis of the force transmission member.

4. The medical rotation mechanism according to claim 3, wherein the driver is configured of an elastic member.

5. The medical rotation mechanism according to claim 4, wherein the plurality of external teeth and the plurality of internal teeth engage with each other in a plurality of locations.

6. The medical rotation mechanism according to claim 4, wherein a cycloid curve or a cycloid parallel curve is formed in the circumferential direction on the internal circumferential surface of the rotation member.

7. The medical rotation mechanism according to claim 4, wherein a trochoid curve or a trochoid parallel curve is formed in the circumferential direction on the internal circumferential surface of the rotation member.

8. A medical rotation mechanism, comprising:
a force transmission member formed in a cylindrical shape and configured to transmit a force;
a driver formed in a cylindrical shape and disposed at an external side of the force transmission member;
a cover having elasticity and configured to cover the external surface of the driver; and
a rotation member formed in a cylindrical shape and disposed at an external side of the driver,
wherein the force transmission member has a cam portion configured to push the driver outwardly in a radial direction, apart of the cam portion in the circumferential direction having a larger length in the radial direction than that of an other part in the circumferential direction of the cam portion,
the driver has a plurality of external teeth which are arrayed in the circumferential direction and movable outwardly in a radial direction,
the rotation member has a plurality of internal teeth arrayed in the circumferential direction on an internal circumferential surface of the rotation member,
the cover is configured to press the plurality of external teeth inwardly in the radial direction,
the cam portion of the force transmission member pushes part of the plurality of external teeth outwardly in the radial direction to make the plurality of external teeth and the plurality of internal teeth to engage with each other, and
the force from the force transmission member is transmitted to the rotation member via the driver to rotate the rotation member.

9. The medical rotation mechanism according to claim 8, wherein a number of plurality of internal teeth is more than a number of plurality of external teeth.

10. The medical rotation mechanism according to claim 9, wherein the force transmission member is configured to transmit the force to the driver by rotating around a longitudinal axis of the force transmission member.

11. The medical rotation mechanism according to claim 10, wherein a rotation axis of the force transmission member coincides with a rotation axis of the rotation member.

12. The medical rotation mechanism according to claim 11, wherein a cycloid curve or a cycloid parallel curve is formed in the circumferential direction on the internal circumferential surface of the rotation member.

13. The medical rotation mechanism according to claim 11, wherein a trochoid curve or a trochoid parallel curve is formed in the circumferential direction on the internal circumferential surface of the rotation member.

14. A medical rotation mechanism, comprising:
a force transmission member formed in a cylindrical shape and configured to transmit a force;
a driver formed in a cylindrical shape and having elasticity, the driver being disposed at an external side of the force transmission member; and
a rotation member formed in a cylindrical shape and disposed at an external side of the driver,
wherein the force transmission member has a cam portion configured to push the driver outwardly in a radial direction, apart of the cam portion in the circumferential direction having a larger length in the radial direction than that of an other part in the circumferential direction of the cam portion,
the driver has a plurality of external teeth which are arrayed in the circumferential direction and movable outwardly in a radial direction,
the rotation member has a plurality of internal teeth arrayed in the circumferential direction on an internal circumferential surface of the rotation member,
the cam portion of the force transmission member pushes part of the plurality of external teeth outwardly in the radial direction to make the plurality of external teeth and the plurality of internal teeth to engage with each other, and
the force from the force transmission member is transmitted to the rotation member via the driver to rotate the rotation member.

15. The medical rotation mechanism according to claim 14, wherein a number of plurality of internal teeth is more than a number of plurality of external teeth.

16. The medical rotation mechanism according to claim 15, wherein the force transmission member is configured to transmit the force to the driver by rotating around a longitudinal axis of the force transmission member.

17. The medical rotation mechanism according to claim 16, wherein a rotation axis of the force transmission member coincides with a rotation axis of the rotation member.

18. The medical rotation mechanism according to claim 17, wherein a cycloid curve or a cycloid parallel curve is formed in the circumferential direction on the internal circumferential surface of the rotation member.

19. The medical rotation mechanism according to claim 17, wherein a trochoid curve or a trochoid parallel curve is formed in the circumferential direction on the internal circumferential surface of the rotation member.
